(19) United States
(12) Patent Application Publication
(10) Pub. No.: US 2020/0368163 A1
(43) Pub. Date: Nov. 26, 2020

(54) ENCAPSULATED WATER-DISPERSIBLE CANNABINOID NANOPARTICLE COMPOSITIONS, METHODS AND SYSTEMS

(71) Applicant: The Trustees of Princeton University, Princeton, NJ (US)

(72) Inventors: Rodney D. Priestley, Princeton, NJ (US); Robert K. Prud'homme, Lawrenceville, NJ (US); Nicholas J. Caggiano, Princeton, NJ (US)

(21) Appl. No.: 16/883,555

(22) Filed: May 26, 2020

Related U.S. Application Data
(60) Provisional application No. 62/852,421, filed on May 24, 2019.

(51) Int. Cl. A61K 9/14; A61K 31/05; A61K 47/38; A61K 9/51

(52) U.S. Cl. CPC A61K 9/14; A61K 31/05; A61K 9/5161; A61K 9/5192; A61K 47/38

(57) ABSTRACT

The present technology relates to encapsulated particles of cannabidiol (CBD) by confined impingement jet (CIJ) mixing or multi-inlet vortex mixing (MIVM). The methods and processes employ intense, turbulent mixing of a solvent stream and an antisolvent stream, to generate self-assembled drug-encapsulating particles stabilized by a polymer. The CBD particles are unexpectedly and advantageously stable in an aqueous environment. They may be subsequently used in the formulation of a variety of products for oral ingestion, including powders for tablets, within creams, or beverage formulations. The resultant products are further highly stable over extended periods of time.

### CROSS-REFERENCE TO RELATED APPLICATIONS

The present application claims priority from pending U.S. Provisional Application No. 62/852,421 filed May 24, 2019, the entirety of which is incorporated by reference herein.

### BACKGROUND

The present technology relates to compositions comprising cannabinoids such as cannabidiol, as well as methods of making such compositions; in particular, methods of producing encapsulated particles of cannabinoids.

Cannabidiol (CBD) is a naturally occurring chemical compound constituting the non-psychoactive portion of the Cannabis sativa plant. CBD is a major non-psychoactive available from the plant; however, there are more than 100 other psychoactives in total (all, including CBD, are collectively referred to herein as cannabinoids). When extracted from the plant source, CBD is in an oil-based form (although portions of it are typically recrystallized during the extraction process), and thus is a highly hydrophobic molecule (log P ˜6) that exhibits poor aqueous solubility. When a drug is taken orally, it must dissolve in the aqueous gastrointestinal environment in order to enter the bloodstream. The poor solubility of CBD leads to decreased oral absorption in the body and consequent limited in vivo bioavailability when CBD is ingested.

Thus, the bioavailability of CBD can be greatly improved though any method that will increase its “solubility” in water.

Attempts have been made to produce ingestible forms of CBD. Some known products include nanoemulsions; however, these are based on oil dispersions, and thus have drawbacks—among them, continued poor solubility and unreliable and inadequate dissolution in the aqueous gastrointestinal environment, unpredictable delivery of the active ingredient, and poor stability over time. Further known “nano-CBD” formulations are largely based on nanomilled CBD—generally, crystalline forms of CBD having reduced crystalline particle size. Many of these exhibit the same problems caused by poor aqueous solubility and dissolution, and consequent insufficient stability. While some attempts have been made to generate amorphous solid dispersions of CBD to avoid the problems of crystalline forms, a concern exists that particles of the CBD can recrystallize.

Therefore, an ongoing need exists for processes for producing stable forms of cannabinoid compositions comprising CBD, as well as resultant formulations that have CBD as an active pharmaceutical ingredient (API) and that are rapidly absorbed in the body; and that exhibit stability and predictability in release of the API.

Advantageous methods of formulating nanoparticles and encapsulates were previously disclosed in U.S. patent application Ser. No. 10/472,071 (now U.S. Pat. No. 8,137,699 to Johnson et al.) and Ser. No. 13/368,888 (now U.S. Pat. No. 9,956,179) and Ser. No. 15/913,294 (U.S. Patent Application Publication No. 2018/0193280); in particular, formulation of nanoparticles through the use of Flash NanoPrecipitation (FNP). However, known FNP methods have, up to this point, worked most successfully with compounds having greater hydrophobicity (log P>6).

Attempts have been made to extract CBD and other cannabinoids from the hemp plant. Some processes merely extract the oil, while more skilled manufacturers can extract the crystalline form. When isolated from the hemp plant in its solid crystalline state, CBD molecules are aligned in an ordered lattice. In contrast, molecules in the amorphous state lack long-range order; these forms having higher energy states tend to lead to increased aqueous solubility, termed “supersaturation.” The methods herein are advantageous, in that they are unexpectedly successful at generating stabilized CBD in the amorphous state as small particles such as nanoparticles, as well as providing rapid dispersibility after formation; thus providing superior formulations.

### SUMMARY

In certain embodiments, the present technology is directed to a method of producing water-dispersible cannabidiol (CBD), comprising the steps of:

(a) providing a solvent stream comprising CBD and a cellulosic amphiphilic polymer;

(b) providing an antisolvent stream comprising water; and

(c) forming a dispersion by mixing the solvent stream and the antisolvent stream and allowing the CBD to precipitate from the solution in the form of nanoparticles that are stabilized in the dispersion by the cellulosic amphiphilic polymer.

In certain embodiments, the present technology is directed to a system for generating water dispersible CBD, the system comprising:

(a) a solvent reservoir, the solvent comprising CBD and a cellulosic amphiphilic polymer;

(b) an antisolvent reservoir, the antisolvent comprising water; and

(c) a mixer having a first inlet port operably connected to the solvent reservoir, a second inlet port operably connected to the anti solvent reservoir, and a mixing chamber operably connected to the first and second inlet ports and to a first outlet port.

In certain embodiments, the present technology is directed to compositions comprising particles formed according to the methods and processes herein; for example, a composition comprising encapsulated nanoparticles of CBD, wherein at least 65% of the particles by weight have a particle size less than 750 nm; wherein the CBD is primarily or substantially entirely in amorphous form; and wherein the composition is optically clear.

### BRIEF DESCRIPTION OF THE DRAWINGS

FIG. 1 shows an exemplary method for forming encapsulated nanoparticles, according to certain embodiments herein.

FIG. 2 shows stability data related to nanoparticles formed according to the methods and processes of certain embodiments herein.

FIGS. 3A to 3B show data regarding the characteristics of resultant particles formed according to the methods and processes of certain embodiments herein.

FIG. 4 shows particle size data related to resultant particles formed according to the methods and processes of certain embodiments herein.

FIGS. 5A and 5B show dissolution data in aqueous conditions of certain compositions discussed herein.

FIGS. 6A through 6D show data on long term stability of exemplary compositions herein.

FIG. 7A shows a schematic of various polymers used in certain embodiments set forth herein.

FIG. 7B shows a scanning electron microscope (SEM) image of an exemplary particle formed according to certain methods and processes discussed herein.

FIG. 8 shows the particle size of various formulations measured over time, using various polymers.

FIGS. 9A through 9C show data for various capsules containing mixtures of cannabinoids.

FIG. 10 shows a plot of transmittance of particle dispersions as a function of particle concentration for certain compositions herein.

### DETAILED DESCRIPTION

All percentages expressed herein are by weight unless otherwise indicated (for example, some percentages herein are by volume, and are indicated as “v/v”). All sizes of particles expressed herein are by length of diameter unless otherwise indicated; further, all sizes, unless otherwise noted, represent the Z-average particle diameter as measured by DLS. All particle size distributions refer to the intensity-weighted distribution (it will be recognized by someone skilled in the art that these DLS measures equivalent hydrodynamic diameters).

As used herein, “monodisperse” refers to particles with a dispersity index less than 0.25.

As used herein, “essentially free of” an ingredient means containing less than 5% of such ingredient. As used herein, “completely free of” an ingredient means containing none of that ingredient.

As used herein, “primarily” means more than 50% of a quantity or value. As used herein, “substantially” means within 5% of a quantity or value—for example, “substantially entirely amorphous” means within 5% of entirely amorphous.

As used herein, “supersaturation” means above the saturation ratio as follows: S=C/C*, where C is the measured concentration and C* is the equilibrium concentration of the crystalline form in the media of interest. Any S value above 1 is supersaturation.

As used herein, “water-dispersible” means having particles small enough to form an optically clear suspension in water.

As used herein, “optically clear” means being transparent to the naked eye, sufficient to make the compositions useful for making formulations for ingestion, such as beverages. In certain embodiments, this can mean having 75% or higher transmittance, or 80% or higher transmittance, at particle concentrations of no more than 0.1 mg/mL. In certain embodiments, compositions according to the present embodiments have 10% or higher transmittance, or 30% or higher transmittance, at particle concentrations no more than 1 mg/mL.

As used herein, “nanoparticle” refers to a small particle that is less than 1060 nm in diameter.

In various embodiments, the compositions formed by the methods and processes herein comprise nanoparticles of less than 1060 nm, less than 750 nm, less than 500 nm, less than 450 nm, less than 400 nm, less than 300 nm, less than 250 nm, less than 200 nm or less than 100 nm in diameter; as well as particles that are deemed sub-micron, that is, less than 1,000 nm; as well as particles that are 1060 nm or greater. That is, while the present disclosure may refer to nanoparticles in certain instances, the methods, processes and systems described are not limited to those that produce only nanoparticles as defined herein, and can be used to make larger particles.

In various embodiments, at least 65% of the particles by weight have a particle size less than 1060 nm; or at least 80% of the particles have a particle size less than 1060 nm; or at least 95% of the particles have a particle size less than 1060 nm as measured by light scattering, microscopy, or other appropriate methods.

In various embodiments, at least 65% of the particles by weight have a particle size less than 500 nm; or at least 80% of the particles have a particle size less than 500 nm; or at least 95% of the particles have a particle size less than 500 nm as measured by light scattering, microscopy, or other appropriate methods.

In various embodiments, at least 65% of the particles by weight have a particle size less than 300 nm; or at least 80% of the particles have a particle size less than 300 nm; or at least 95% of the particles have a particle size less than 300 nm as measured by light scattering, microscopy, or other appropriate methods.

In various embodiments, at least 65% of the particles by weight have a particle size of 100 to 300 nm; or at least 80% of the particles have a particle size less of 100 to 300 nm; or at least 95% of the particles have a particle size of 100 to 300 nm as measured by light scattering, microscopy, or other appropriate methods.

In certain embodiments, the methods herein involve utilizing a confined impinging jet (CIJ) mixer (also referred to interchangeably as “confined impingement jet” or “jets”) to produce encapsulated particles of the active cannabidiol (CBD) using a polymer such as an amphiphilic polymer—that is, a batch process approach. In certain embodiments, the methods herein utilize a multi-inlet vortex mixer (MIVM)—that is, a continuous process approach. In either case, the approaches employ the polymer to encapsulate active ingredients (also referred to herein as “drug” or API or active pharmaceutical ingredient). Such approaches represent new mechanisms and modes of operation for such mixers.

In certain embodiments, the methods herein employ rapid, turbulent mixing of a solvent and antisolvent to generate essentially self-assembled encapsulating nanoparticles. During this intense mixing, CBD molecules can precipitate from solution, and are stabilized by the presence of a polymer while in the mixer—that is, the polymer attaches to the surface of the molecules and acts as a stabilizer. The resultant core-shell particles (core comprising CBD, shell comprising polymer) are stable in an aqueous environment, and can be subsequently processed for use in the formulation of consumer products including powders, tablets, creams and other forms for consumer use. In certain embodiments, because of their visual clarity, they can be formulated into beverages or other oral dosage forms for which optical clarity is desirable.

### Cannabidiol (CBD) and Cannabinoids

Cannabidiol (CBD) is a naturally occurring chemical compound which, along with more than 100 other cannabinoids, constitute the non-psychoactive portion of the Cannabis sativa plant. In addition to the cannabinoids, the plant also includes the psychoactive compound tetrahydrocannabinol (THC).

The present technology is directed to, in certain embodiments, compositions and methods that include pure CBD or substantially pure CBD, as well as mixtures that include CBD and any other cannabinoids extracted from the Cannabis sativa plant; but do not include THC. These include, for example, CBD isolate (purified CBD) and broad spectrum CBD (CBD with other phytocannabinoids or extracts such as terpenes, but no THC). Further, all mention of “CBD” or “cannabidiol” in the present disclosure refers to not only pure or substantially pure CBD, but also to any composition that comprises CBD but includes other compositions that may form part of the extracted compound from the plant, for example, other cannabinoids.

In certain embodiments, the CBD to be subjected to the processes and methods herein is initially obtained in crystalline form, or primarily in crystalline form. As used herein, “primarily” means more than 50% in crystalline form; in various embodiments, the CBD in the solvent stream is at least 60%, at least 70%, at least 80% or at least 90% in crystalline form. In other embodiments, the CBD in the solvent stream is initially obtained in amorphous form, or primarily in amorphous form, or at least 60%, at least 70%, at least 80% or at least 90% in amorphous form. The CBD can be combined with a polymer to provide a solvent stream, as described herein and in the Examples.

In the various embodiments discussed herein, the CBD that is put into the methods, systems and processes can be obtained commercially, and can be in crystalline form, amorphous form, or a combination thereof.

### Solvent Stream and Antisolvent Stream

In certain embodiments of the processes herein, a solvent stream containing CBD and a polymer is mixed with an antisolvent stream.

In certain embodiments, the solvent stream can comprise one or more ethereal solvents such as, e.g., tetrahydrofuran (THF); or any other solvent such as acetone, alcohol or the like. Any organic solvent miscible with water can be used as a solvent in the methods and processes herein.

In certain embodiments, the solvent stream further comprises one or more additional compounds that can be mixed with the CBD or other cannabinoids to form the core of the nanoparticles as the end product. Because CBD can be sensitive to oxidation, it has herein been investigated whether antioxidants would be beneficial in further stabilizing the CBD.

Examples of such compounds include, but are not limited to: an antioxidant such as vitamin E; or any other vitamin (e.g., vitamin A, vitamin C, beta-carotene), provitamin, antioxidant (e.g., lutein, lycopene, selenium, zeaxanthin) or compound that can prevent oxidation, recrystallization or degradation of the CBD; or otherwise add further stability to the CBD. Other examples include permeability enhancers or terpenes. In certain embodiments, a co-core comprising vitamin E has been found to be particularly beneficial, as it was observed herein that vitamin E can both increase the hydrophobicity of the core, and also slow down or prevent oxidation of the CBD. In various embodiments, a suitable compound such as a drug that is desired to be co-delivered with the CBD can be included, as well as any other enhancing product such as an intestinal permeabilizer, e.g., capric acid or a salt thereof; whether the additional compound is beneficial for protecting the CBD, enhancing the bioavailability or release of the CBD from the final dosage form, or desired to be delivered along with the CBD for any other reason.

In certain embodiments, the antisolvent stream comprises water, including water alone or any aqueous media, or any combination thereof. In certain embodiments, the antisolvent stream comprises aqueous media such as an aqueous buffer, e.g., a citrate buffer. In certain embodiments, the citrate buffer further provides the additional benefit of protecting the CBD—i.e., it can act as an antioxidant.

### Polymers

As mentioned above, in certain embodiments, the CBD can be combined with a polymer to provide a solvent stream, as described herein and in the Examples. In certain embodiments, during a process herein, as the solvent stream and antisolvent stream are combined, the CBD particles precipitate out of the solution, and the polymers attach to the CBD particles during this process, stabilizing the CBD particles.

In certain embodiments, a polymer herein is a cellulosic amphiphilic polymer. Certain cellulosic amphiphilic polymers useful for the methods herein can include a functionalized hydroxypropyl methylcellulose molecule. In certain embodiments, a useful amphiphilic polymer herein is a hydroxypropyl methylcellulose that contains one or more succinyl or acetyl substituents. An exemplary useful amphiphilic polymer is a hypromellose acetate succinate (HPMCAS). HPMCAS is a naturally derived, semi-synthetic, amphiphilic soluble polymer that assists in the formation of solid dispersions, and also has the unexpected and added benefit of inhibiting API recrystallization in solution. In certain embodiments, this promotes supersaturation of the drug—that is, the ability to achieve higher concentrations of the drug when released into the body (and hence increased bioavailability) as compared to the crystalline form. In various embodiments herein, methods that include only an HPMCAS as the sole polymer, or as the polymer along with any other polymer, yield particles with increased stability over time.

In other embodiments, the compositions produced herein can show increased bioavailability or release of cannabinoid composition when redispersed, e.g., at least a 10%, at least a 20%, or at least a 30% increase in bioavailability or release. In certain embodiments, the increase in bioavailability or release is not to the point of supersaturation, or is to the point of supersaturation, e.g., 1 to 10 or 1 to 50 or 1 to 100.

In various embodiments, any HPMCAS can be used in the methods herein—including, but not limited to, different grades such as HPMCAS 716, HPMCAS 912, and HPMCAS 126 (the numbers referring to the ratio of succinyl and acetyl substituents on the HPMC backbone). Useful amphiphilic polymers for the present methods can be obtained, e.g., under the trade name Aquasolve™ from Ashland Inc. (Covington, Ky., U.S.A.) or under the trade name Agoat™ from Shin-Etsu Chemical Co., Ltd. (Tokyo, Japan).

Other polymers may be employed for the methods herein—other amphiphilic polymers including but not limited to: block copolymers, zein (a prolamine protein found in corn), lecithin (phospholipids often extracted from soy); as well as amphiphilic acrylate copolymers; PVP-VA; or amphiphilic copolymers containing any of the following: polyethylene glycol (PEG), polyvinyl caprolactam, or polyvinyl acetate; as well as any amphiphilic polymeric compound that can achieve the desired end products discussed herein.

### Mixing and Encapsulation Processes

The processes herein are, in certain embodiments, drawn to methods of making particles such as nanoparticles by precipitating the molecules from a water-miscible organic solvent, and then drying and pulverizing the precipitate to form the particles. Advantageously, the particles in the embodiments herein can be stabilized by the presence of the polymer, as discussed in further detail below.

In certain embodiments, the present technology is directed to methods of producing water-dispersible cannabidiol (CBD), comprising mixing the solvent stream and the antisolvent stream using a Confined Impinging Jets (CIJ) mixer or a Multi-Inlet Vortex Mixer (MIVM) and allowing the CBD to precipitate from the solution; wherein the CBD is stabilized in the dispersion by the presence of the cellulosic amphiphilic polymer. Thus, in certain embodiments, a solvent stream containing acetone or THF along with dissolved drug and a stabilizing amphiphilic polymer, is impinged with an antisolvent (in most embodiments, water). During this intense and turbulent mixing of solvent and antisolvent streams, CBD molecules precipitate from solution. Put another way, the addition of antisolvent in the mixing chamber creates supersaturated drug concentrations in the mixer; this leads to precipitation or phase separation of the drug into particles. The growth of such precipitates is arrested by the attachment of a stabilizing amphiphilic polymer.

Flash NanoPrecipitation (FNP) is a process used to fabricate core-shell nanoparticles in which a hydrophobic core is stabilized by a polymeric shell. The FNP process involves rapid, turbulent mixing of solvent and antisolvent streams, which are brought together in a mixer. Precipitation of the hydrophobic core occurs during mixing of the two streams as a result of high concentrations within the mixing chamber, followed by attachment of the polymeric stabilizer to the surface of the core. FNP is scalable and in various embodiments may be performed as either a batch or continuous process to produce monodisperse, uniform particles.

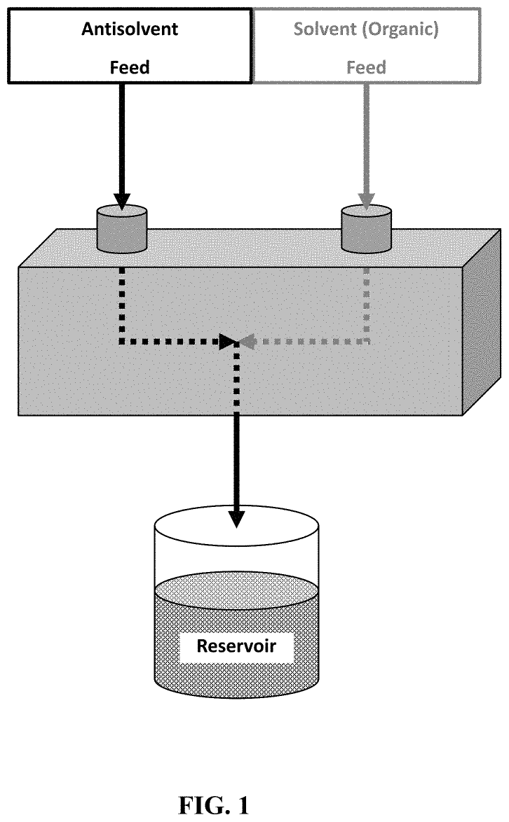

In certain embodiments, for example, as shown in FIG. 1, a process herein involves an FNP process conducted at small scale using a CIJ mixer. The mixer can have two inlet (feed) streams, which in certain embodiments, are of equal volume or substantially equal volume. The solvent, or organic, stream can be comprised of polymer and drug (core) dissolved in a water-miscible organic solvent. The antisolvent stream can be aqueous, e.g., plain DI water or any desirable buffer or salt solution or combination thereof, including, for example a citrate buffer. In various embodiments, the pH of the anti solvent stream is in the range of 5 to 6, or 6 to 7 or 6 to 8, for example, 5 to 5.5 or 6 to 6.5. In certain embodiments, the presence of a citrate buffer is further advantageous, as the HPMCAS polymer is pH sensitive; therefore, it can be possible to control the size and stability of the particles with the antisolvent stream. It has been found herein that generally, a lower pH will result in larger particles as end products; conversely, a higher pH will result in smaller particles. Thus, the size of particles desired can be controlled at least in part by the pH of the antisolvent stream. In certain embodiments, the pH of the solvent stream is in the range of 5 to 5.5 or 5 to 6 (corresponding approximately to the pKa of the acidic groups in the polymer, e.g., the succinate groups on the HPMCAS).

The solvent and antisolvent streams can be fed to the mixer via, for example, manually or automatically actuated syringes or any similar form of solvent reservoir or antisolvent reservoir (for example, a holding tank or any pump such as a syringe pump that can control flowrate). In certain embodiments, following the precipitation of the hydrophobic core and surface attachment of the polymeric stabilizer in the mixing chamber, the particles can flow into an aqueous quenching reservoir to further dilute the concentration of organic solvent to prevent Ostwald ripening.

At this point, the particles can be filtered in certain embodiments, but in other embodiments the particles are too small to be effectively separated and recovered by a filter, so no filter is present. After exiting the mixer, the particles are typically dispersed in a mostly aqueous media. This dispersion can then be dried, for example, via spray drying, lyophilization, drying under vacuum or freeze drying to obtain a dry, primarily dry or substantially dry powder. In certain embodiments hydroxypropyl methylcellulose (HPMC) or mannitol, or a combination of both, can be used as a matrix or dispersing agent in the spray drying step.

In various embodiments, the mass ratio of the matrix to the nanoparticles can be varied to achieve desirable results. That is, the Mass Ratio=(Mass of Matrix):(Mass of Nanoparticles).

Almost any water-soluble polymer, sugar or salt can be used as the matrix material. The goals of the matrix material can include any of the following: (1) the matrix material can protect the nanoparticles during the drying step, to prevent aggregation or fusing of the particles to each other; or (2) the matrix can dissolve rapidly away in water, thus releasing the nanoparticles. Examples of useful matrix materials include any of the following: a water soluble cellulose, e.g., HPMC, CMC, croscarmellose sodium; a sugar, e.g., sucrose, mannitol, trehalose, sorbitol; a salt, e.g., buffer salt (including citrate), or any other edible ionic salt. In certain embodiments, a matrix material is chosen for its ability to avoid undesirable taste effects.

In certain embodiments, the mass ratio of the matrix material to CBD nanoparticles is 50:1 to 2:1; or 25:1 to 2:1, or 20:1 to 2:1 or 10:1 to 2:1 or 5:1 to 2:1. It has been found herein that a ratio in these ranges delivers optimal benefit in providing nanoparticles that are rapidly dispersible in an aqueous medium, and have substantially the same diameter as the original particles that have been produced by a method or process herein.

In certain embodiments, a CIJ process herein uses 0.5 mL of antisolvent, 0.5 mL of solvent, and 4 mL of reservoir media, for a total batch size of 5 mL and final organic solvent concentration of 5 to 25%, or 10 to 20%, or 10% or 15% or 20% (all of the percentages of solvent concentration in his paragraph being by volume (v/v)).

In certain embodiments, the processes herein can be conducted in a Multi-Inlet Vortex Mixer (MIVM) rather than a CIJ mixer. A primary difference is that while a CIJ typically has two inlet streams and can operate in a batch process configuration, an MIVM typically has up to four (it may have, for example, two or four inlet streams), and can operate in a continuous process configuration. In certain embodiments, a process herein can be a process incorporating some aspects of the FNP process but conducted in an MIVM mixer. In such case, the MIVM can be fed by programmable syringe pumps and can handle a wide range of flowrates in any combination that gives the desired output concentration of organic solvent by volume as described above, so as to provide for homogeneous kinetics. For example, the total flowrate could be 50 to 600 mL/min, or 60 to 500 mL/min, or 75 to 400 mL/min, or 100 to 300 mL/min, or 110 to 250 mL/min, or 120 to 200 mL/min, or 130 to 175 mL/min. One or more of the individual streams could have a flowrate of 10 to 200 mL/min, or 15 to 150 mL/min, or 15 to 100 mL/min or 15 to 50 mL/min.

For further example, in certain embodiments an MIVM mixer according to the technology herein can have a “higher” throughput configuration of 67/200/200/200 mL/min (solvent/antisolvent/antisolvent/antisolvent); or a “lower” throughput configuration of 16/48/48/48 mL/min (solvent/antisolvent/antisolvent/antisolvent); or a still lower throughput configuration of 8/24/24/24 mL/min (solvent/antisolvent/antisolvent/antisolvent).

In an exemplary embodiment, a typical process can include one solvent feed stream at 10 to 20 mL/min, plus three antisolvent streams, each at 40 to 50 mL/min. Similar to the CIJ with reservoir, this can be used, in certain embodiments, to produce a final organic solvent concentration of 5 to 25%. In other embodiments, the antisolvent streams need not be divided evenly; that is, two or more can have different flowrates.

In certain embodiments, the technology herein is directed to the user's ability to adjust the parameters of the methods and processes described herein in order to produce encapsulated nanoparticles having a desired size and optical clarity. For example, while experimental results showed that the parameters of 75% core CBD at 10 mg/mL total solids (where total solids here refers to the mass of CBD+mass of polymer in the organic stream, which is the total mass of everything dissolved in the solvent stream) and 50% core CBD at 15 mg/mL total solids were found to result in increased loading (that is, increased amount of CBD in the core), these also resulted in particles size of 250 to 300 nm that were less optically clear (more opaque). For certain embodiments, these parameters could be more desirable, and therefore, a user can optimize features such as CBD loading, particle size, efficiency of the method and appearance of the particles, depending on desired end products. Thus, in certain embodiments, the processes and methods herein are directed to methods of controlling the size of the particles depending on the unique needs of the consumer or the particular application for which the processes are being utilized.

As discussed above, CBD is known to exhibit poor aqueous solubility. However, these encapsulated nanoparticles may be able to increase the solubility, and hence bioavailability, of CBD in vivo. The resultant dried nanoparticles can be, in certain embodiments, in the form of a powder that can then be used as an oral solid dosing form (e.g. tablet, capsule, sublingual tablet, orally disintegrating tablet) or remain suspended in a liquid for potential use as a food/beverage additive, i.e., water dispersible or water soluble CBD.

In certain embodiments, the present technology is directed to systems that comprise any one or more of the following, for example, a solvent reservoir, an antisolvent reservoir, a mixing chamber (adapted for either a batch process configuration or a continuous process configuration); one or more pumps for conveying the solvent or antisolvent streams from their respective reservoirs into the mixing chamber at user-determined and user-controlled flowrates; and a reservoir connected to the outlet of the mixing tank that is used to quench the particles that are produced. In various embodiments, the mixing chamber is configured to mix the solvent and antisolvent when both enter the mixing chamber, in a batch process configuration; or is configured to mix the solvent and antisolvent when both enter the mixing chamber, in a continuous process configuration. In certain embodiments, the system further comprises a reservoir connected to the first outlet port, such that the mixed solvent and antisolvent exiting the mixing chamber is collected in the reservoir.

### Advantages

The methods, systems and products discussed herein exhibit numerous advantages. Among them are highly and unexpectedly stable products. In various embodiments, the resultant nanoparticles contain unexpectedly high amounts (loadings) of CBD—in various embodiments, greater than 50%, greater than 60% or greater than 70% by weight. PXRD of the resultant products shows that the CBD is at least partially, and in certain embodiments substantially entirely, or entirely, in amorphous form. Release studies of the resultant products show a relatively rapid burst of CBD release within about 15 minutes of dosing the nanoparticle powder into aqueous dissolution media.

As discussed above, the nanoparticles created by the processes and methods herein are extremely stable. In certain embodiments, the nanoparticles contain the amorphous form of CBD (as opposed to the crystalline form). For example, in certain embodiments, the particles precipitated out of the combined solvent and antisolvent streams is primarily in crystalline form. As used herein, “primarily” means more than 50% in amorphous form; in various embodiments, the CBD particles at least 60%, at least 70%, at least 80% or at least 90% in amorphous form. In addition, the amorphous drug is found to provide particularly fast delivery of the CBD and related cannabinoids herein to the body of the patient. This can overcome the drawbacks found in other forms of CBD and methods of delivering them to users.

Further, the resultant nanoparticles from the methods, processes and systems herein can be rapidly dispersible or dispersed (also referred to interchangeably herein as “redispersible” or “redispersed”), when contacted with a solvent such as a liquid. This can make them desirable for delivery of CBD in liquid formulations, such as beverages. For example, known powders resulting from known methods (including FNP processes known to this point) have been relatively difficult to disperse, frequently requiring vigorous mixing or sonication of 15 minutes or more. In contrast, the present methods, processes and systems can yield particles that can completely, or substantially completely, disperse when contacted with a solvent, with nothing more than gentle stirring (or in certain embodiments, gentle shaking, or no stirring or shaking at all) within 30 seconds, within 1 minute, within 2 minutes, within 5 minutes or within 10 minutes after contact with the solvent, e.g., after mixing and stirring into the solvent. In various embodiments, at least 50%, at least 75% or at least 80% of the compound disperses within the aforementioned time period. In certain embodiments, this dispersion occurs without the necessity of changing the temperature (e.g., heating or cooling) of the nanoparticles or the solvent—that is, the dispersion can occur at any temperature, including room temperature, a lower temperature or a higher temperature.

Still other advantages can be seen from the methods and processes herein. For example, the methods and processes herein can, in certain embodiments, shield the API from light exposure and degradation in a way that other known allegedly “water soluble” CBD formulations (such as emulsions, solid dispersions, and milled powders) cannot.

The processes and methods herein are scalable and can be used to power large scale production. CBD nanoparticles encapsulated with a natural, GRAS, polymer has been prepared by FNP. The particles are stable in an aqueous environment, and can be subsequently used in the formulation of powders for tablets or within creams. The dispersions can also be formulated into beverages in which optical clarity is paramount.

Several batches of nanoparticles have been produced and characterized to determine size (over time) and zeta potential. The stability of the particles can be desirable to further processing (such as filtering, lyophilization or spray drying) or for stability in suspension over time (e.g. in a beverage product or other oral dosage form).

### EXAMPLE 1

Ten samples of product were tested as follows:

Stock solutions of CBD and HPMCAS 126 were prepared in the organic solvent of choice, as listed in Table 1. The stock solutions were mixed to prepare a solution with the desired concentrations of CBD (expressed as “core concentration”) and polymer in the solvent for a particular batch. A summary of the batches produced, and the characteristics of each, is shown in Table 1. Total solids means core concentration in solvent+polymer concentration in solvent. Target core % (also expressed as “% core”) means core concentration divided by total solids.

TABLE 1

| | | | | | Core | Polymer | | |
|---|---|---|---|---|---|---|---|---|
| | | | | | Concentration | Concentration | Total | Target |
| | | | | | in Solvent | in Solvent | Solids | Core |
| Sample | Core | Polymer | Solvent | Antisolvent | (mg/mL) | (mg/mL) | (mg/mL) | % |
| F1 3-2 | CBD | HPMCAS 126 | THF | citrate 10 mM pH 5.2 | 2.5 | 7.5 | 10 | 25% |
| F2 3-2 | CBD | HPMCAS 126 | THF | citrate 10 mM pH 5.2 | 5 | 5 | 10 | 50% |
| F3 3-2 | CBD | HPMCAS 126 | THF | citrate 10 mM pH 5.2 | 7.5 | 2.5 | 10 | 75% |
| F4 3-2 | CBD | HPMCAS 126 | THF | citrate 10 mM pH 5.2 | 3.75 | 11.25 | 15 | 25% |
| F5 3-2 | CBD | HPMCAS 126 | THF | citrate 10 mM pH 5.2 | 7.5 | 7.5 | 15 | 50% |
| F6 3-2 | CBD | HPMCAS 126 | THF | citrate 10 mM pH 5.2 | 3.75 | 11.25 | 15 | 75% |
| F7 3-2 | CBD | HPMCAS 126 | THF | citrate 10 mM pH 5.2 | 5 | 15 | 20 | 25% |
| F8 3-2 | CBD | HPMCAS 126 | THF | citrate 10 mM pH 5.2 | 10 | 10 | 20 | 50% |
| F9 3-2 | CBD | HPMCAS 126 | THF | citrate 10 mM pH 5.2 | 15 | 5 | 20 | 75% |
| F10 3-2 | CBD | HPMCAS 126 | THF | citrate 10 mM pH 6.2 | 5 | 5 | 10 | 50% |

For each sample, a volume of 0.5 mL of solvent solution was loaded into a syringe, and 0.5 mL of antisolvent was loaded into a separate syringe. The syringes were fitted into the inlet ports of the CIJ mixer. A vial containing the reservoir bath comprised of 4 mL of antisolvent was placed underneath the outlet of the mixer. For samples F1 through F9 in Table 1, the antisolvent comprised 10 mM of a citrate buffer at a pH of 5 to 6. For sample F10, the citrate buffer was at a pH of 6 to 7.

The syringes were rapidly actuated, injecting both the solvent and antisolvent solutions into the mixing chamber at substantially the same time. Inside the chamber, the CBD precipitated or phase separated from solution. The growth of CBD domains was arrested by the surface attachment of HPMCAS, thus forming stabilized core-shell particles. The particles then exited the mixer into the quenching reservoir. Particle size was measured by Dynamic Light Scattering (DLS).

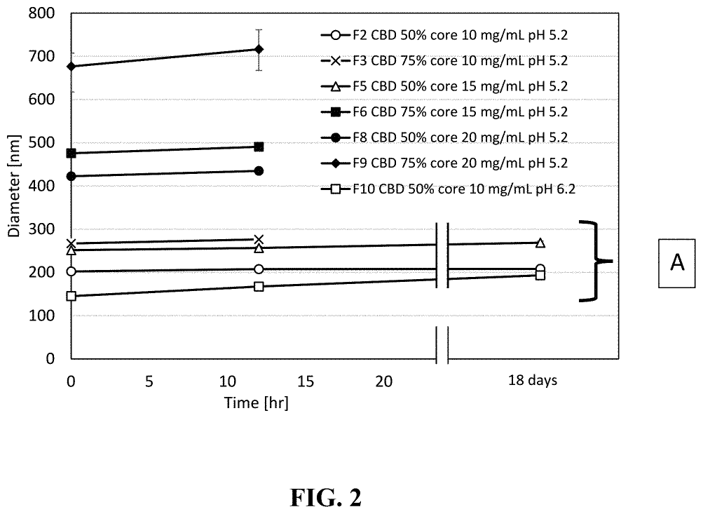

The particles were then spray dried with an HPMC E3 and mannitol matrix/dispersing agent, resulting in nanoparticles that were found to be on average between 100 and 800 nm in diameter. As shown in FIG. 2 (designated as region “A”), the most size stable particles were in the range of 150 to 300 nm, and exhibited size stability—that is, their size stayed within a value of no more than 30% or 20% or 10% of their size at time of formation, during a period of time of at least 10 hours, at least 12 hours, or at least 24 hours, at least 48 ours, or at least 7 days, or from 7 to 18 days, or up to 18 days or more. In this particular experiment, these particles were F2, F3, F5 and F10. As can be seen in FIG. 2, in certain embodiments, as used herein, “size stable” means that the majority of these particles (in various embodiments, at least 65%, at least 80% or at least 95% of these particles) stay within at least 30% of their size (i.e., diameter) between the time they were formed from a method or process herein, and a measured end time as set forth in the present disclosure. Similarly, in certain embodiments, at least 65%, at least 80% or at least 95% of these particles stay within least 10% or at least 20% of their size at formation, over any given 5 hour time window or longer, or any 10 hour time window or longer, or any 18 hour time window or longer, or any 24 hour time window or longer. Put another way, the products herein are size stable over long periods of time and do not significantly agglomerate or aggregate (and thus do not gain size).

As used herein, when referring to measurement of diameter of particles “at the time they were formed” or “at the time of formation” or similar language, this means at the time a particular particle has precipitated from the solution and the fully or substantially fully encapsulated nanoparticle has been formed. However, in certain embodiments, the particles formed herein are able to maintain their size, substantially their size or within 30%, 20% or 10% of their size, from as early as the time they are precipitated from the solution, from the time they are spray dried (turned into powder), and even from the time they are redispersed into a consumer product, e.g., a liquid form such as a beverage or other oral form.

Comparative results showed that CBD encapsulated with a PS-b-PEG block copolymer did not yield size-stable nanoparticles in a similar way, as discussed in Example 5 and elsewhere in the present disclosure.

### EXAMPLE 2

A solvent stream comprising 5 mg/mL of CBD and 5 mg/mL of HPMCAS in THF (similar to Formulation F2 from Example 1) was filled into a glass syringe, which was mounted in a syringe pump. Similarly, DI water antisolvent was filled into three additional glass syringes, which were mounted into a separate syringe pump. The four syringe outlets were connected to the four inlet ports of the MIVM using suitable tubing and fittings. The syringe pumps were programmed to feed the solvent solution at a rate of 16 mL/min, while each of the three antisolvent streams was fed at 48 mL/min. This resulted in a total flow of 160 mL/min of feed solution through the mixer, at a final composition of 10% v/v organic solvent. No reservoir was used.

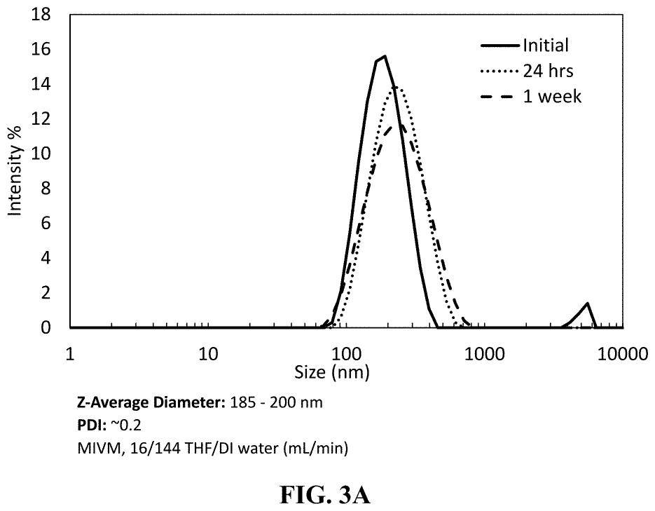

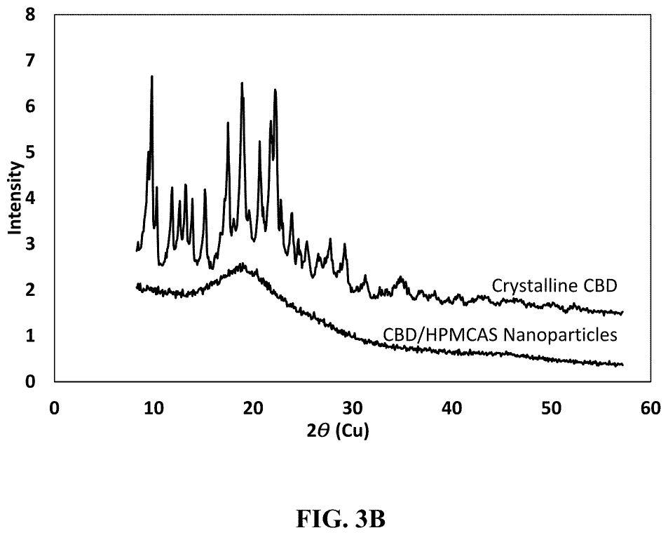

An aliquot of particle dispersion from MIVM outlet was collected and retained, and particle size characterized by DLS at three timepoints: initial, 24 hr, and 1 week. Results are shown in FIG. 3A, and indicate that formulation of nanoparticles of CBD can be run as a continuous process in the MIVM, producing similarly sized, uniform particles that show good stability.

The rest of the dispersion was spray dried. HPMC E3 was added at a 1:1 ratio (E3 mass:nanoparticle mass) before spray drying. A dry powder was obtained by the spray drying. The powder was characterized by PXRD. Results are shown in FIG. 3B, and indicate that the particles are amorphous after spray drying.

### EXAMPLE 3

Formulations made according to the methods herein were tested for redispersion of the nanoparticles after the spray drying step. In this case, mannitol was added at a 50:1 ratio (mannitol mass:nanoparticle mass) before spray drying. A dry powder was obtained by spray drying. The powder was added to DI water and gently stirred for 30 seconds until the powder visually appeared dispersed. An aliquot was removed, and particle size was characterized by DLS.

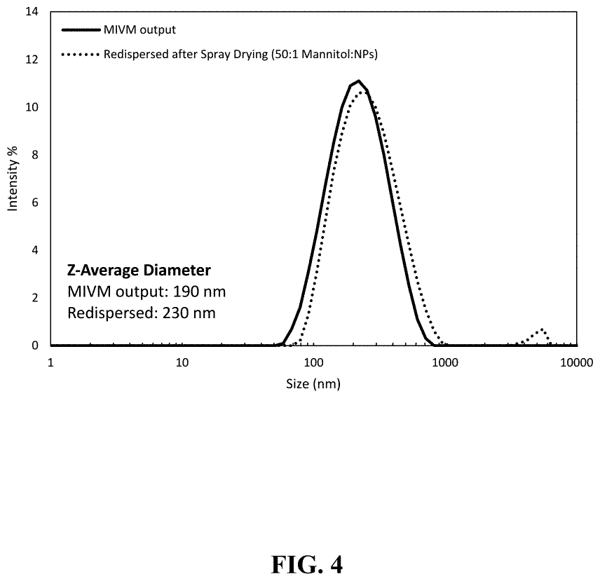

Results are shown in FIG. 4. They show that substantially all of the redispersed particles had a size within 20% of their size at time of output from the MIVM (approximately 40 nm).

### EXAMPLE 4

A nanoparticle formulation comprised of CBD and HPMCAS, at a CBD core percentage of 50% (similar to Formulation F2 from Example 1, and from the same batch of formulation made in Example 2) was formed in accordance with certain embodiments of the present technology. The spray drying matrix was 1:1 HPMC E3. The resultant particles were then dosed into fed-state simulated intestinal fluid (FeSSIF) that was obtained and prepared according to manufacturer instructions (Biorelevant, Ltd.).

Release studies were conducted at a temperature of 37° C. using a temperature-controlled water bath. Two vials of media were prepared, each containing approximately 10 mL of prepared and pre-warmed FeSSIF. CBD crystalline powder was added to one vial. To the other vial the spray-dried CBD nanoparticle powder was added at an equivalent CBD dose. The CBD nanoparticle powder in the study had a CBD content of approximately 22%, quantified by HPLC, while the CBD crystals were substantially 100% CBD. Therefore, enough nanoparticle powder was added such that the equivalent CBD mass was equal to the mass of the crystalline CBD.

Aliquots were taken from each vial at 1, 5, 10, 15, 30, 60 and 120 minutes.

Aliquots were centrifuged to pellet any undissolved particles, and the supernatant was removed and lyophilized to yield a dry powder.

The powder was reconstituted in a solvent mixture of 1:4 THF:acetonitrile, which was analyzed by HPLC. Concentration of released CBD was quantified using a calibration curve.

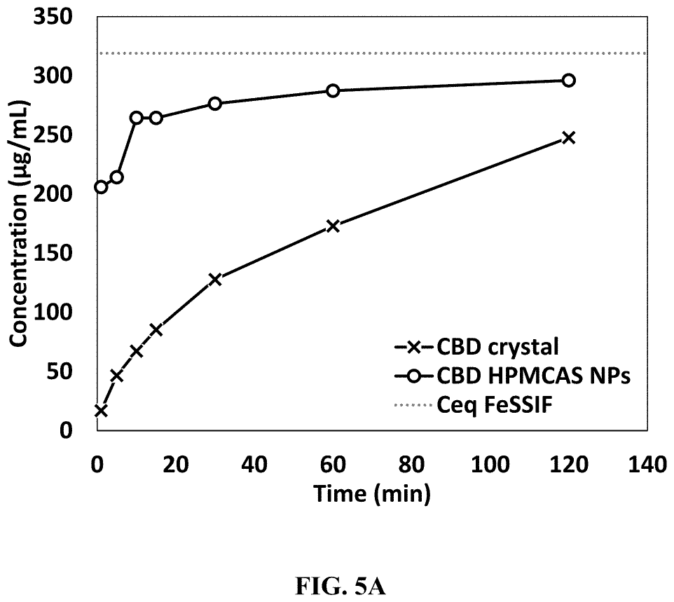

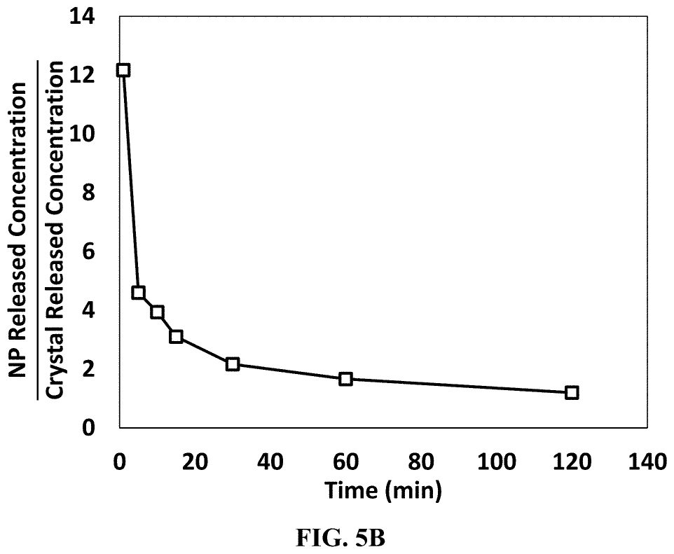

Results showed rapid burst release of nanoparticles within about 15 minutes. FIG. 5A shows a graph of the results, indicating the time period of 0 up to 120 minutes, and also including a control line for the equilibrium solubility (C_{eq}) of CBD in FeSSIF media at 37° C. (previously measured).

FIG. 5B further shows the ration of concentrations—that is, the concentration released from the nanoparticles divided by the concentration released from the crystal control sample. As shown there, the ratio is much greater than 1 at very early timepoints, indicating that the nanoparticle formulation offers much faster burst release of CBD than the crystal. That is, when dosed into simulated intestinal fluid, the nanoparticle formulation releases higher CBD concentrations faster, which is beneficial in an oral formulation. Over time, the crystal does begin to dissolve, and the ratio approaches 1. However, as shown from the curve of the graph, for at least the first 10 to 20 minutes, the formulations discussed herein exhibit much faster release than the known crystalline forms of CBD (which are on the order of 100 microns).

### EXAMPLE 5

Comparative tests were made between the nanoparticles produced with the methods herein, and those produced using CBD as a core with block copolymers (some with a vitamin E co-core) instead of the amphiphilic polymers discussed in the embodiments herein.

The control formulations tested were as follows, in Table 2:

TABLE 2

| | | | | | | Core | Co-core | Polymer | | |
|---|---|---|---|---|---|---|---|---|---|---|
| | | | | | | Concentration | Concentration | Concentration | Total | Target |
| | | | | | | in Solvent | in Solvent | in Solvent | Solids | Core |
| Sample | Core | Co-core | Polymer | Solvent | Antisolvent | (mg/mL) | (mg/mL) | (mg/mL) | (mg/mL) | % |
| F1 3-17 | CBD | — | PS-b-PEG | THF | DI water | 2.5 | — | 7.5 | 10 | 25% |
| F2 3-17 | CBD | Vitamin | PS-b-PEG | THF | DI water | 2.5 | 2.5 | 5 | 10 | 25% |
| | | E | | | | | | | | |
| F3 3-17 | CBD | — | PS-b-PEG | THF | DI water | 5 | — | 5 | 10 | 50% |
| F4 3-17 | CBD | — | PCL-b-PEG | THF | DI water | 2.5 | — | 7.5 | 10 | 25% |
| F5 3-17 | CBD | Vitamin | PCL-b-PEG | THF | DI water | 2.5 | 2.5 | 5 | 10 | 25% |
| | | E | | | | | | | | |
| F6 3-17 | CBD | — | PCL-b-PEG | THF | DI water | 5 | — | 5 | 10 | 50% |
| F7 3-17 | Vitamin | — | PS-b-PEG | THF | DI water | 5 | — | 5 | 10 | 50% |
| | E | | | | | | | | | |
| F8 3-17 | Vitamin | — | PCL-b-PEG | THF | DI water | 5 | — | 5 | 10 | 50% |
| | E | | | | | | | | | |

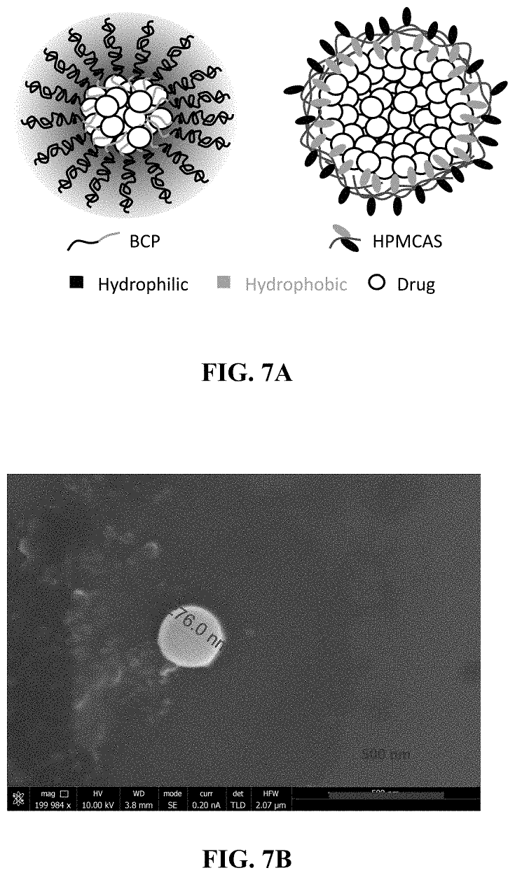

Results suggested that PS-b-PEG (1.6k-5k) was ineffective at stabilizing particles with pure, or substantially pure, CBD cores. Formulations comprising pure, or substantially pure, CBD cores with PS-b-PEG polymers, i.e., F1 and F3, produced particles that were unstable in solution, and that observably began aggregating within hours after production (see FIG. 7B). PS-b-PEG did produce more stable particles through the addition of vitamin E as a more hydrophobic co-core in combination with CBD (i.e., F2). This formulation, comprised of 25% CBD, 25% vitamin E, and 50% PS-b-PEG, produced particles at a size of approximately 100 nm. PCL-b-PEG (5k-5k) was able to form size stable CBD particles, low loading (25%), resulting in a particle size of approximately 50 nm (Formulation F4). PCL-b-PEG particles at higher CBD loading (50%) were not stable and instead rapidly aggregated (Formulation F6). Stable particles of approximately 100 nm in size were produced when vitamin E was included as a co-core in addition to CBD (Formulation F5).

Put another way, PS-b-PEG was found to be a poor stabilizer, as it did not produce stable particles with pure, or substantially pure, CBD cores (e.g., 25 and 50%). The vitamin E co-cores were found to help produce size-stable particles. PCL-b-PEG was a better stabilizer, but still not fully satisfactory, as it could stabilize pure, or substantially pure, CBD particles only at low core loadings (e.g., 25%), so it was not efficient. There, the vitamin E co-cores were also found to help produce size-stable particles.

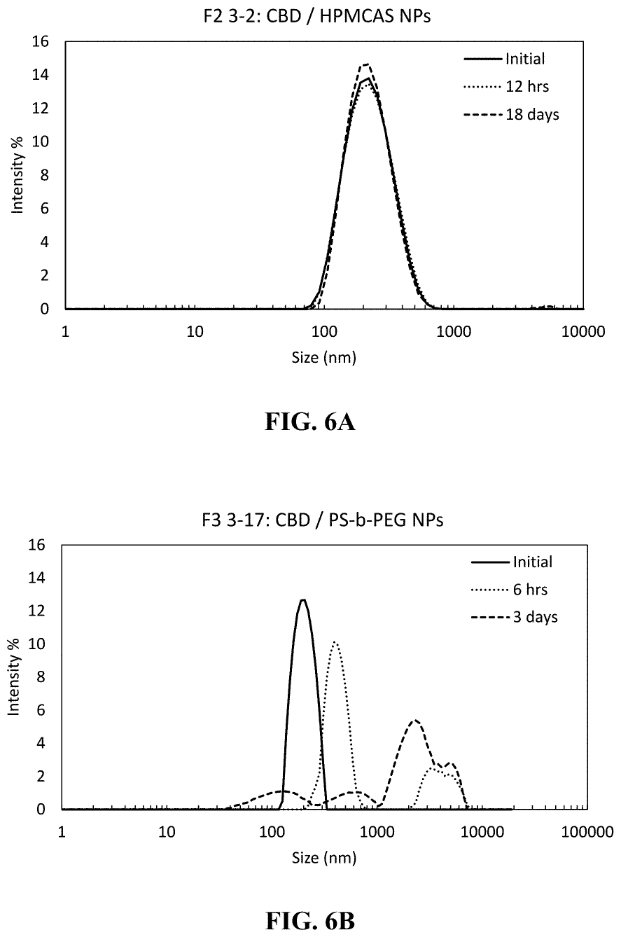

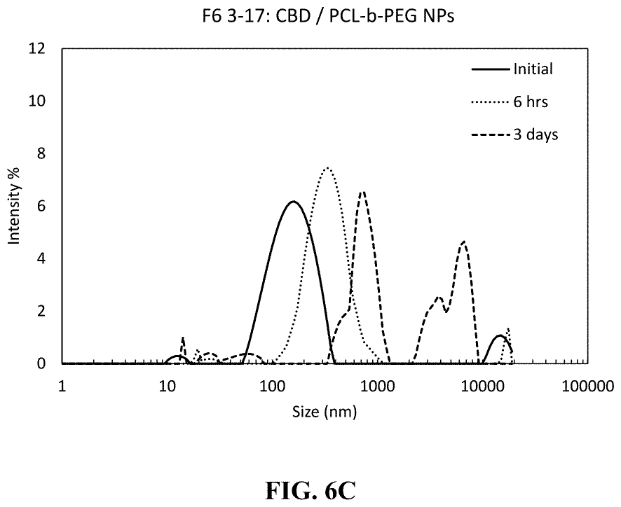

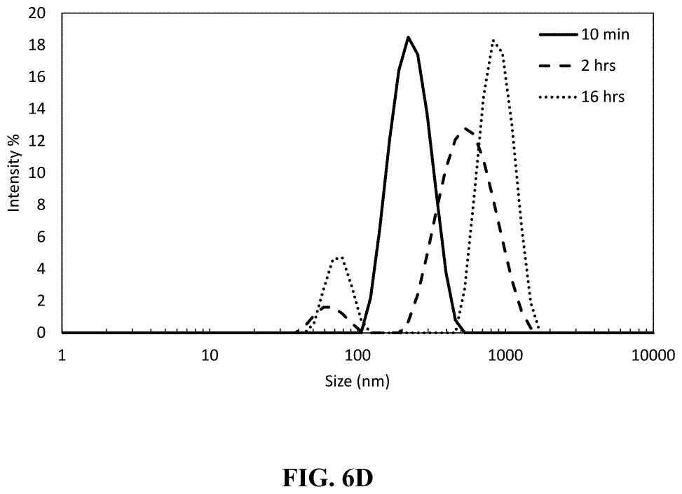

FIGS. 6A through 6D show further comparisons of the data generated from methods including a block copolymer core (some with a vitamin E co-core) instead of the exemplary amphiphilic polymers discussed herein. It can be seen that the polymer-stabilized nanoparticles produced in accordance with the embodiments herein exhibit superior stability and preservation of size and integrity of the particles over time.

FIG. 6A shows a plot of sample F2 3-2 from Table 1. The solvent stream was CBD 5 mg/mL+HPMCAS polymer 5 mg/mL in THF. The antisolvent stream was citrate buffer 10 mM ph 5.2. Results showed a very stable PSD over a duration of up to 18 days or more.

FIG. 6B shows a plot of sample F3 3-17 from Table 2. The solvent stream was CBD 5 mg/mL+PS-b-PEG polymer (Mn. 1.6k-5k) 5 mg/mL in THF. The antisolvent stream was DI water. Results showed that the PSD rapidly deteriorated over 6 hours (likely due to particle aggregation).

FIG. 6C shows a plot of sample F6 3-17 from Table 2. The solvent stream was CBD 5 mg/mL+PCL-b-PEG polymer (5k-5k) 5 mg/mL in THF. The antisolvent stream was DI water. Results showed that the PSD rapidly deteriorated over 6 hours (likely due to particle aggregation).

FIG. 6D shows another example of the behavior of unstable particles formed using a block copolymer stabilizer. The solvent stream was CBD 5 mg/mL+PS-b-PEG polymer (1.6k-5k) 5 mg/mL in THF. The antisolvent stream was DI water. Here it can be seen that monodisperse particles are formed initially, but after only 2 hours the distribution becomes bimodal as the particles undergo Ostwald ripening and aggregation.

FIGS. 7A and 7B show a schematic comparison of block copolymer (BCP) versus HPMCAS nanoparticle morphology, as well as a scanning electron microscopy (SEM) image of an HPMCAS/CBD nanoparticle prepared using the methods herein. It is herein surmised that particles stabilized by BCPs rely on point anchoring of the hydrophobic block in the core and the formation of a PEG brush. In contrast, HPMCAS chains form an exterior shell layer by orienting the hydrophobic acetyl functional groups inward towards the core, with the hydrophilic succinate groups oriented outwards. The HPMCAS shell layer is thought to more effectively hinder outward diffusion of the core than the BCP brush, thus enabling higher core loadings and providing more robust size stability over time. The SEM image shows that the particles formed by the methods herein are substantially spherical.

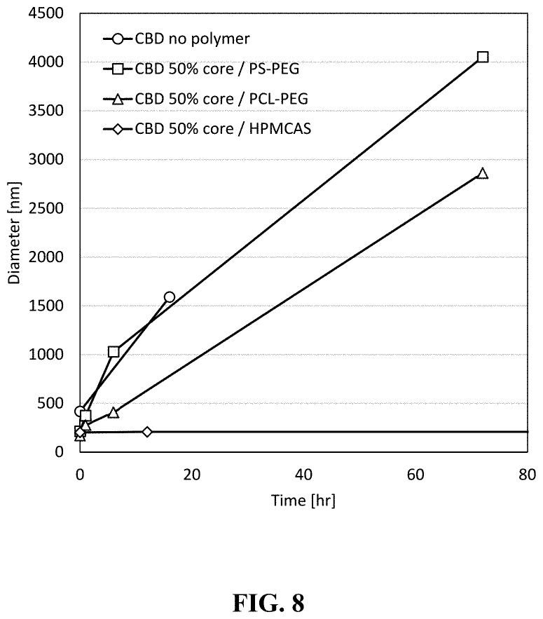

FIG. 8 shows the comparison of diameter as a function of time. Formulations are all 5 mg/mL CBD+5 mg/mL polymer in THF (“CBD no polymer” is only 5 mg/mL CBD in THF). The last three formulations are F3 3-17, F6 3-17, F2 3-2.

It is seen that a formulation comprised of CBD, without any polymeric stabilizer, initially forms particles around 500 nm in size which rapidly aggregate in solution.

Similarly, formulations comprised of 50% CBD and 50% block copolymer stabilizer (PS-PEG or PCL-PEG) form particles that aggregate within hours after formation.

In contrast, a formulation comprised of 50% CBD and 50% HPMCAS exhibits excellent size stability over time, maintaining a size of approximately 200 nm over a period of 18 days (last data point not shown).

It is concluded that amphiphilic polymers such as HPMCAS provide better particle stabilization when as the encapsulating shell layer of the particles, as compared to the block copolymers tested. Additionally, CBD on its own was shown not to form stable particles.

### EXAMPLE 6

Formulations according to the present methods, processes and systems were tested to see how the transmittance of particle dispersions can vary as a function of particle concentration. Two samples were prepared; samples F1 and F2 were both CBD/HPMCAS nanoparticles, each comprised of 50% CBD and 50% HPMCAS. All formulation and process parameters were identical, except for the pH of the citrate buffer antisolvent stream which was varied to tune particle size. The initial average particle size of sample F1 was approximately 220 nm, while that of sample F2 was approximately 190 nm. Particles were originally produced at a concentration of 1 mg/mL during the process of formation, and were further diluted with additional antisolvent solution. Transmittance was measured using an Anton Paar Litesizer 500. All measurements were conducted at a temperature of 25° C. Transmittance is reported as a percentage, with the transmittance value of a pure DI water reference sample representing 100%.

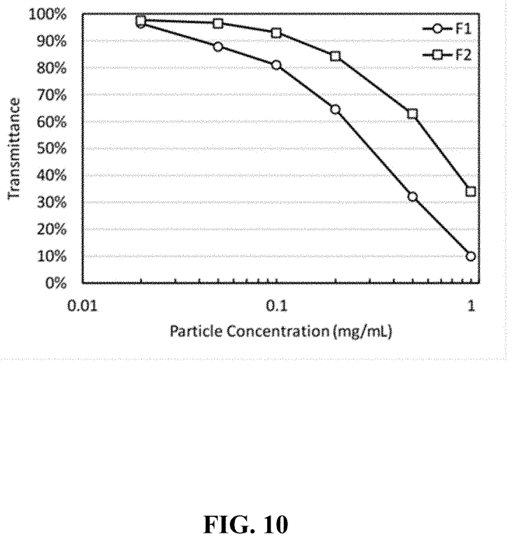

Results are shown in FIG. 10. It can be seen that the optical transmittance of sample F2 was generally greater than that of F 1, as a result of the lower particle size achieved by modulating the antisolvent pH during FNP. At concentrations potentially desirable for beverage formulations in certain embodiments herein, sample F2 was nearly optically transparent, with transmittance values above 80% and at some points above 90%. Sample F1 was also fairly transparent at similar concentrations, with transmittance values above 80%. Note that in certain embodiments, 0.05 to 2 mg/mL, 0.05 to 1.5 mg/mL or 0.1 mg/mL are optimal beverage concentrations, based on a normal dose of CBD being about 15 mg per 12 oz beverage can. In various embodiments, antisolvent pH: F1=pH 5 to 6 or 5.2; F2=pH 6 to 7 or 6.2.

### EXAMPLE 7

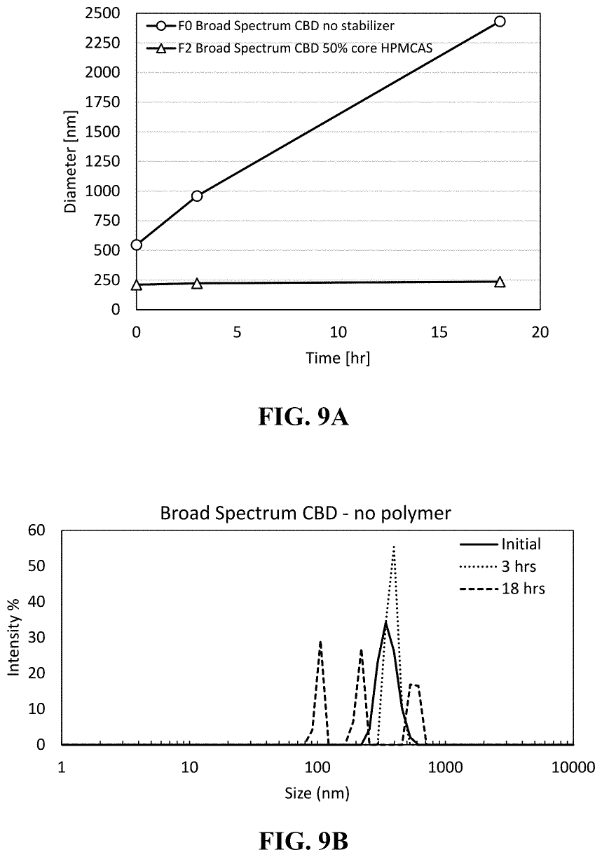

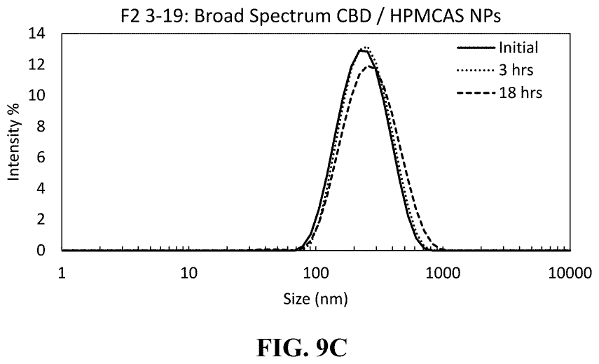

FIGS. 9A to 9C illustrate further evidence that the methods, processes and systems herein can also be used to encapsulate mixtures comprised of CBD and other non-THC cannabinoids. Specifically, these Broad Spectrum CBD distillates tested that had 80 to 90% CBD (in the case of these Figures, ˜83% CBD). FIG. 9A shows a comparison between a formulation comprising the CBD distillate without polymer stabilizer (FO), and a formulation comprising the CBD distillate along with HPMCAS as the stabilizing polymer (5 mg/mL each in THF, DI water antisolvent). It is seen that the particles which include the HPMCAS stabilizing shell are size stable at a large range of diameters, e.g., at 200 to 300 nm (in certain embodiments, approximately 220) nm for up to 18 hours or more. In comparison, the formulation without any polymer rapidly aggregates within hours after formation. FIG. 9B shows the particle size distribution of formulation F0 over time; FIG. 9C shows the particle size distribution for F2 over time.

The methods herein have been found to yield many advantages—among them, in certain embodiments the resultant CBD encapsulated particles have an average size of 150 to 300 nm, or 175 to 250 nm, or 195 to 225 nm. In certain embodiments, the resultant particles are optically clear when diluted, and are sufficient to make the compositions useful for formulations for ingestion, such as beverages.

In addition, control studies were conducted with block copolymer stabilizers alone, and results show that the polymers used herein in conjunction with the methods and processes herein can stabilize particles with higher CBD loading. Thus, the methods, processes and systems herein can provide increased efficiency as well as better size stability over time. Size stability is an important advantage, in two periods of time in particular:

(1) The nanoparticles resulting from the methods, processes and systems herein are advantageously able to maintain stability (e.g., size stability) for periods of time after they are precipitated from the solution in the mixing chamber, and before they are spray dried, since the processes herein can generally produce particles at a much faster rate and greater throughput than the spray drying can be completed. Thus, in various embodiments, at least 65%, at least 75% or at least 85% of the resultant nanoparticles in the embodiments herein can maintain their size (or at least stay within 30% or within 20% or within 10% of their size when precipitated from the solution in the mixing chamber) for a period of at least 6 hours, at least 12 hours, at least 18 hours or at least 24 hours after their formation, before being spray dried.

(2) The encapsulated nanoparticles resulting from the methods, processes and systems herein are advantageously able to maintain stability (e.g., size stability) for periods of time after they are spray dried (that is, when they are in powder form) but before they are reconstituted into a consumer composition of choice, e.g., an oral composition such as a beverage. In certain embodiments, the spray dried powders of the compositions herein could be provided to formulators as raw products for formulation into consumer products such as beverages. These can advantageously maintain their stability during this period. Thus, in various embodiments, at least 65%, at least 75% or at least 85% of the resultant nanoparticles in the embodiments herein can maintain their size (or at least stay within 30% or within 20% or within 10% of their size upon formation into powder form for a period of at least 10 hours, at least 12 hours, or at least 24 hours, at least 48 ours, or at least 7 days, or from 7 to 18 days, or up to 18 days or more, or even up to 1 month or 2 months or more during this time period.

(3) The encapsulated nanoparticles resulting from the methods, processes and systems herein are advantageously able to maintain stability (e.g., size stability) for periods of time after they are constituted into a consumer composition of choice, e.g., an oral composition such as a beverage. Long term stability is particularly important for oral formulations including CBD or cannabinoids, where the particles could potentially sit for months before being consumed. Thus, in various embodiments, at least 65%, at least 75% or at least 85% of the resultant nanoparticles in the embodiments herein can maintain their size (or at least stay within 30% or within 20% or within 10% of their size upon formation into powder form for a period of at least 10 hours, at least 12 hours, or at least 24 hours, at least 48 ours, or at least 7 days, or from 7 to 18 days, or up to 18 days or more, or even up to 1 month or 2 months or more.

The methods, processes, systems and formulations discussed herein are easily scalable and can be used to power large-scale production of CBD products. Further, as mentioned above, the ease and rapidity with which the compositions formed herein can be dispersed in a solvent is absolutely unexpected and surprising, and constitute a remarkable achievement over the known art. In certain embodiments, the particles formed herein are monodisperse. In various embodiments, they exhibit a dispersity index of less than 0.5, less than 0.3, less than 0.25, less than 0.2, or less than 0.15. One of ordinary skill in the art knows that the scale is 0 to 1; lower values are more desirable, and mean there is less variation in particle size. In various embodiments, at least 65%, at least 70%, at least 80% or at least 85% of the particles herein are monodisperse; or exhibit a given dispersity index as set forth above.

Although the present technology has been described in relation to embodiments thereof, these embodiments and examples are merely exemplary and not intended to be limiting. Many other variations and modifications and other uses will become apparent to those skilled in the art. The present invention should, therefore, not be limited by the specific disclosure herein, and can be embodied in other forms not explicitly described here, without departing from the spirit thereof.

We claim:

1. A method of producing water-dispersible cannabidiol (CBD), the method comprising the steps of:
(a) providing a solvent stream comprising CBD and a cellulosic amphiphilic polymer;
(b) providing an antisolvent stream comprising water; and
(c) forming a dispersion by mixing the solvent stream and the antisolvent stream to form a solution, and allowing the CBD to precipitate from the solution in the form of nanoparticles that are stabilized in the dispersion by the cellulosic amphiphilic polymer.

2. The method of claim 1, wherein the particles of CBD that precipitate from the solution have an average particle size of less than 1060 nm in diameter.

3. The method of claim 2, wherein the particles of CBD that precipitate from the solution have an average particle size of less than 500 nm in diameter.

4. The method of claim 3, wherein the particles of CBD that precipitate from the solution have an average particle size of less than 250 nm in diameter.

5. The method of claim 1, wherein the CBD in the resultant dispersion is primarily in amorphous form.

6. The method of claim 1, wherein the cellulosic amphiphilic polymer is a functionalized hydroxypropyl methylcellulose molecule.

7. The method of claim 6, wherein the hydroxypropyl methylcellulose molecule contains one or more succinyl and acetyl substituents.

8. The method of claim 1, wherein the solvent stream comprises an alcohol or acetone.

9. The method of claim 1, wherein the solvent stream comprises an ethereal solvent.

10. The method of claim 9, wherein the ethereal solvent is tetrahydrofuran (THF).

11. The method of claim 1, wherein the antisolvent stream comprises an aqueous citrate buffer.

12. The method of claim 1, further comprising spray drying the dispersion to produce nanoparticles in powder form that, when redispersed, maintain an average diameter within 30% of their diameter at the time of formation.

13. A system for generating water dispersible CBD, the system comprising:
(a) a solvent reservoir, the solvent comprising CBD and a cellulosic amphiphilic polymer;
(b) an antisolvent reservoir, the antisolvent comprising water; and
(c) a mixer having a first inlet port operably connected to the solvent reservoir, a second inlet port operably connected to the antisolvent reservoir, and a mixing chamber operably connected to the first and second inlet ports and to a first outlet port.

14. The system of claim 13, wherein the mixing chamber is configured to mix the solvent and antisolvent when both enter the mixing chamber, in a batch process configuration.

15. The system of claim 13, wherein the mixing chamber is configured to mix the solvent and antisolvent when both enter the mixing chamber, in a continuous process configuration.

16. The system of claim 13, further comprising a reservoir connected to the first outlet port, such that the mixed solvent and antisolvent exiting the mixing chamber is collected in the reservoir.

17. A composition comprising encapsulated nanoparticles of CBD, wherein at least 65% of the particles by weight have a particle size less than 750 nm; wherein the CBD is primarily in amorphous form; and wherein the composition is optically clear.

18. The composition of claim 17, wherein at least 65% of the particles maintain an average diameter within 30% of their diameter at their formation, for at least 6 hours after their formation.

19. The composition of claim 17, in the form of a powder.

20. The composition of claim 19, wherein the composition substantially disperses within 2 minutes of contact with a solvent.

21. The composition of claim 17, wherein at least 65% of the particles are monodisperse.

22. The composition of claim 17, wherein the composition has 75% or higher transmittance at a particle concentration of no more than 0.1 mg/mL.
